(19) United States
(12) Patent Application Publication
(10) Pub. No.: US 2019/0175471 A1
(43) Pub. Date: Jun. 13, 2019

(54) Personal Care Composition

(71) Applicant: Colgate-Palmolive Company, New York, NY (US)

(72) Inventors: Sandra Wadeer, Flanders, NJ (US); Joan Gambogi, Hillsborough, NJ (US); Christine Boyke, Somerset, NJ (US)

(73) Assignee: Colgate-Palmolive Company, New York, NY (US)

(21) Appl. No.: 15/835,959

(22) Filed: Dec. 8, 2017

(51) Int. Cl. A61K 8/37; A61Q 15/00; A61K 8/58; A61K 8/86; A61K 8/34; A61K 8/92; A61K 8/362

(52) U.S. Cl. CPC A61K 8/37; A61Q 15/00; A61K 8/58; A61K 8/86; A61K 2800/30; A61K 8/922; A61K 8/342; A61K 8/362; A61K 8/34

(57) ABSTRACT

An antiperspirant composition including an antiperspirant active, a surfactant, a gelling agent, and an emollient, wherein the emollient is one of PPG-3 isostearyl methyl ether, PPG-3 benzyl ether myristate, PPG-3 benzyl ether ethylhexanoate, isoamyl cocoate, and diethylhexyl carbonate.

### BACKGROUND

Personal care compositions, such as antiperspirant or deodorant compositions and dual purpose antiperspirant-deodorant compositions, may be used to reduce body odor. Antiperspirant or deodorant compositions may be applied to axillary (underarm) regions to limit perspiration, limit or kill odor-causing bacteria in the region, or apply a fragrance. Antiperspirant or deodorant compositions may be delivered topically as roll-on, gel, stick, cream, or aerosol formulations.

However, depending on their ingredients, the personal care composition may also leave an unsightly white residue on the skin or clothing after being applied.

Accordingly, there is a desire for antiperspirant or deodorant compositions that are formulated to reduce or eliminate the amount of white residue left after application.

### BRIEF SUMMARY

This section is intended merely to introduce a simplified summary of some aspects of one or more embodiments of the present disclosure. Further areas of applicability of the present invention will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by an antiperspirant composition, including an antiperspirant active; a surfactant; a gelling agent; and an emollient, wherein the emollient comprises at least one of PPG-3 isostearyl methyl ether, PPG-3 benzyl ether myristate, PPG-3 benzyl ether ethylhexanoate, isoamyl cocoate, and diethylhexyl carbonate, and wherein the antiperspirant composition comprises 1% or less of cyclomethicone, based on a total weight of the antiperspirant composition.

In another embodiment, the antiperspirant composition is substantially free of cyclomethicone.

In another embodiment, the antiperspirant composition includes from 10 weight % to 30 weight % of the antiperspirant active; from 1.0 weight % to 15 weight % of the surfactant; from 20 weight % to 30 weight % of the gelling agent; and from 0.1 weight % to 50 weight % of the emollient.

In another embodiment, the antiperspirant active includes aluminum zirconium tetrahydrex glycine.

In another embodiment, the gelling agent includes at least one of hydrogenated castor oil, hydrogenated soy bean oil, and stearyl alcohol.

In another embodiment, the emollient further includes at least one of C12-15 alkyl benzoate, PPG-14 butyl ether, and palm kernel oil.

In another embodiment, the surfactant includes PEG-8 distearate.

The foregoing and/or other aspects and utilities embodied in the present disclosure may be achieved by a cyclomethicone-free antiperspirant composition, including an antiperspirant active; a surfactant; a gelling agent; and a cyclomethicone replacement emollient, wherein the cyclomethicone replacement emollient consists essentially of at least one of PPG-3 isostearyl methyl ether, PPG-3 benzyl ether myristate, PPG-3 benzyl ether ethylhexanoate, isoamyl cocoate, diethylhexyl carbonate, and mixtures thereof, and wherein the antiperspirant composition comprises 1% or less of cyclomethicone, based on a total weight of the antiperspirant composition.

In another embodiment, the cyclomethicone-free antiperspirant composition comprises 0% cyclomethicone.

In another embodiment, the antiperspirant composition includes from 10 weight % to 30 weight % of the antiperspirant active; from 1.0 weight % to 15 weight % of the surfactant; from 20 weight % to 30 weight % of the gelling agent; and from 0.5 weight % to 50 weight % of the emollient, and from 10 weight % to 20 weight % of the cyclomethicone replacement emollient.

In another embodiment, the cyclomethicone replacement emollient consists essentially of PPG-3 isostearyl methyl ether, consists essentially of PPG-3 benzyl ether myristate, consists essentially of PPG-3 benzyl ether ethylhexanoate, consists essentially of isoamyl cocoate, or consists essentially of diethylhexyl carbonate.

The foregoing and/or other aspects and utilities embodied in the present disclosure may be achieved by a method for decreasing the visibility of an antiperspirant composition after application, the method including adding a cyclomethicone replacement emollient to the antiperspirant composition, wherein the cyclomethicone replacement emollient comprises at least one of PPG-3 isostearyl methyl ether, PPG-3 benzyl ether myristate, PPG-3 benzyl ether ethylhexanoate, isoamyl cocoate, and diethylhexyl carbonate, and wherein the antiperspirant composition comprise 1% or less cyclomethicone, based on a total weight of the antiperspirant composition.

In another embodiment, the antiperspirant is substantially free of cyclomethicone.

In another embodiment, the antiperspirant composition includes from 10 weight % to 20 weight % of the cyclomethicone replacement emollient.

In another embodiment, the method further includes adding from 10 weight % to 30 weight % of an antiperspirant active to the antiperspirant composition; adding from 1.0 weight % to 15 weight % of a surfactant to the antiperspirant composition; and adding from 20 weight % to 30 weight % of the gelling agent to the antiperspirant composition; wherein adding the cyclomethicone replacement emollient includes adding from 10 weight % to 20 weight % of the cyclomethicone replacement emollient.

### DETAILED DESCRIPTION

Reference will now be made in detail to the various embodiments in the present disclosure. The embodiments are described below to provide a more complete understanding of the components, processes, compositions, and apparatuses disclosed herein. Any examples given are intended to be illustrative, and not restrictive. However, it will be apparent to one of ordinary skill in the art that the invention may be practiced without these specific details. In other instances, well-known methods, procedures, and components have not been described in detail so as not to unnecessarily obscure aspects of the embodiments.

Throughout the specification and claims, the following terms take the meanings explicitly associated herein, unless the context clearly dictates otherwise. Phrases such as “in some embodiments” and “in an embodiment” as used herein do not necessarily refer to the same embodiment(s), though they may. Furthermore, the phrases “in another embodiment” and “in certain embodiments” as used herein do not necessarily refer to a different embodiment, although they may. As described below, various embodiments may be readily combined, without departing from the scope or spirit of the present disclosure.

As used herein, the term “or” is an inclusive operator, and is equivalent to the term “and/or,” unless the context clearly dictates otherwise. The term “based on” is not exclusive and allows for being based on additional factors not described, unless the context clearly dictates otherwise. In the specification, the recitation of “at least one of A, B, and C,” includes embodiments containing A, B, or C, multiple examples of A, B, or C, or combinations of A/B, A/C, B/C, A/B/B/B/B/C, A/B/C, etc. In addition, throughout the specification, the meaning of “a,” “an,” and “the” include plural references. The meaning of “in” includes “in” and “on.”

It will also be understood that, although the terms first, second, etc. may be used herein to describe various elements, these elements should not be limited by these terms. These terms are only used to distinguish one element from another. For example, a first object, component, or step could be termed a second object, component, or step, and, similarly, a second object, component, or step could be termed a first object, component, or step, without departing from the scope of the invention. The first object, component, or step, and the second object, component, or step, are both, objects, component, or steps, respectively, but they are not to be considered the same object, component, or step. It will be further understood that the terms “includes,” “including,” “comprises” and/or “comprising,” when used in this specification, specify the presence of stated features, steps, operations, elements, and/or components, but do not preclude the presence or addition of one or more other features, steps, operations, elements, components, and/or groups thereof. Further, as used herein, the term “if” may be construed to mean “when” or “upon” or “in response to determining” or “in response to detecting,” depending on the context.

All physical properties that are defined hereinafter are measured at 20° to 25° Celsius unless otherwise specified.

When referring to any numerical range of values herein, such ranges are understood to include each and every number and/or fraction between the stated range minimum and maximum, as well as the endpoints. For example, a range of 0.5-6.0% would expressly include all intermediate values of, for example, 0.6%, 0.7%, and 0.9%, all the way up to and including 5.95%, 5.97%, and 5.99%, among many others. The same applies to each other numerical property and/or elemental range set forth herein, unless the context clearly dictates otherwise.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether or not “about” is used in conjunction therewith.

Unless otherwise specified, all percentages and amounts expressed herein and elsewhere in the specification should be understood to refer to percentages by weight. The amounts given are based on the active weight of the material.

With regard to procedures, methods, techniques, and workflows that are in accordance with some embodiments, some operations in the procedures, methods, techniques, and workflows disclosed herein may be combined and/or the order of some operations may be changed.

Many personal care compositions, such as deodorants or antiperspirants, include cyclomethicones as an emollient or skin conditioning agent. Cyclomethicones may also be used to reduce the tackiness or stickiness associated with some personal care formulations. However, the inventors have surprisingly discovered new cyclomethicone replacements that function as emollients, while also decreasing the amount of white residue left on skin or clothing after application of a personal care formulation.

Accordingly, the present disclosure provides a personal care composition formulated to reduce the amount of white residue left on skin or clothing after application. As used herein, the term “antiperspirant composition” is used to describe personal care compositions that function as antiperspirant compositions, deodorant compositions, or as dual purpose antiperspirant-deodorant compositions.

In certain embodiments, the personal care composition is embodied as an antiperspirant composition including an emollient, a gelling agent, a surfactant, a preservative, and an antiperspirant active.

The antiperspirant composition may be provided as a solid stick composition. The solid stick composition may be manufactured using conventional methods. For example, the ingredients of a solid stick antiperspirant composition may be combined and heated to melt the components (perhaps excluding inert fillers), and the melted components may then be mixed. Typically, volatile materials, such as fragrances, are incorporated at latter stages to avoid volatilization or evaporation. After mixing, the molten composition can be poured directly into dispensers or molds, where the composition cools and hardens into a solid, and the dispenser can be capped to preserve the solid product until use.

The antiperspirant composition includes one or more emollients. For example, the antiperspirant composition may include volatile and non-volatile emollients in any desired amount to achieve a desired emollient effect. Several emollients are known in the art and are used to impart a soothing effect on the skin. Examples of non-volatile emollients include non-silicone and silicone emollients. Examples of non-volatile, non-silicone emollients include C12-15 alkyl benzoate. A non-volatile silicone material can be a polyethersiloxane, polyalkyarylsiloxane or polyethersiloxane copolymer. An illustrative non-volatile silicone material is phenyl trimethicone. Non-limiting examples of emollients can be found in U.S. Pat. No. 6,007,799. Examples include, but are not limited to, PPG-14 butyl ether, PPG-15 stearyl ether, PPG-3 myristyl ether, stearyl alcohol, stearic acid, glyceryl monoricinoleate, isobutyl palmitate, glyceryl monostearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl) succinate, isopropyl myristate, isopropyl palmitate, isopropyl stearate, octacosanol, butyl stearate, glyceryl monostearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, alkyl benzoate, hydrocyethyl stearate amide, and hydrogenated polyisobutene.

In one embodiment, the antiperspirant composition includes palm kernel oil as an emollient.

In other embodiments, the emollient is selected from linear silicones, cyclic silicones, hydrocarbons, polyhydroxy alcohols having more than 3 carbon atoms, liquid or solid polyalkyleneglycol ethers containing a polypropylene glycol (PPG) moiety and terminating in an alkyl ether, and combinations thereof. In another embodiment, the emollient is a nonvolatile silicone, such as dimethiconol or a longer chain dimethicone.

In one embodiment, the antiperspirant composition includes from about 0.1% to about 50% emollients, based on a total weight of the antiperspirant composition. In other embodiments, the antiperspirant composition may include from about 25 weight % to about 45 weight % emollients or from about 30 weight % to about 40 weight % emollients.

In certain embodiments, the antiperspirant composition is formulated to be substantially free or free of cyclomethicones. For example, the antiperspirant composition may include less than 5 weight % cyclomethicone, based on a total weight of the antiperspirant composition, or less than 4 weight %, less than 3 weight %, less than 2 weight %, or less than 1 weight % cyclomethicone.

In other embodiments, the antiperspirant composition contains no cyclomethicones and/or is considered to be cyclomethicone-free.

As used herein, the term “cyclomethicone” refers to the group of methyl siloxanes or cyclic polydimethylsiloxane polymers with low viscosity and high volatility commonly used as skin emollients in personal care formulations. Cyclomethicones may include hexamethylcyclotrisiloxane (CAS: 541-05-9), octamethylcyclotetrasiloxane (CAS: 556-67-2), decamethylcyclopentasiloxane (CAS: 541-02-6), and dodecamethylcyclohexasiloxane (CAS: 540-97-6).

Unless otherwise indicated, as used herein, “cyclomethicone-free” and “free of cyclomethicones” means that cyclomethicone is not purposefully added to the antiperspirant composition and is only present, if at all, in trace amounts or as an impurity. As used herein, “trace amounts” means 0.1 weight % or less. As used herein, the term “completely free” means that the antiperspirant composition does not comprise cyclomethicone. That is, the antiperspirant composition comprises 0 weight % cyclomethicone. For example, the antiperspirant composition may comprise 0 weight % or only trace amounts of decamethylcyclopentasiloxane. In another embodiment, the antiperspirant composition includes 0 weight % or only trace amounts of cyclic-silicones.

In certain embodiments, the antiperspirant composition includes one or more emollients that replace cyclomethicone. These cyclomethicone replacement emollients may provide the antiperspirant composition with a skin conditioning emollient that helps to maintain a soft, smooth, and pliable appearance of the skin. In some embodiments, the cyclomethicone replacements are configured to remain on the skin surface or in the stratum corneum and act as lubricants, to reduce flaking, and to improve the skin's appearance.

For example, the antiperspirant composition may include cyclomethicone replacement emollients such as PPG-3 isostearyl methyl ether, PPG-3 benzyl ether myristate, PPG-3 benzyl ether ethylhexanoate, and mixtures thereof. PPG-3 isostearyl methyl ether is available commercially as Arlamol LST, and PPG-3 benzyl ether myristate and PPG-3 benzyl ether ethylhexanoate are commercially available as, respectively, Crodamol STS and Crodamol SFX, from Croda International, Snaith, U.K.

In certain embodiments, the cyclomethicone replacement emollients may also include isoamyl cocoate, diethylhexyl carbonate, and mixtures thereof. Isoamyl cocoate and diethylhexyl carbonate are commercially available as, respectively, Tegosoft AC and Tegosoft DEC from Evonik Industries, Essen, Germany. In other embodiments, the antiperspirant composition may include any combination of the cyclomethicone replacement emollients listed above. In certain embodiments, the cyclomethicone replacement emollients are limited to one or more of PPG-3 isostearyl methyl ether, PPG-3 benzyl ether myristate, PPG-3 benzyl ether ethylhexanoate, isoamyl cocoate, and diethylhexyl carbonate.

The antiperspirant composition may include from about 10% to about 20% cyclomethicone replacement emollients based on a total weight of the antiperspirant composition. Alternatively, the antiperspirant composition may include from about 12 weight % to about 18 weight % cyclomethicone replacement emollients. In one embodiment, the antiperspirant composition includes about 13.40 weight % cyclomethicone replacement emollients. For example, the antiperspirant composition may include from about 12 weight % to about 13.40 weight % PPG-3 benzyl ether myristate. Alternatively, the antiperspirant composition may include about 13.40 weight % PPG-3 isostearyl methyl ether, about 13.40 weight % PPG-3 benzyl ether ethylhexanoate, about 13.40 weight % isoamyl cocoate, or about 13.40 weight % diethylhexyl carbonate.

The antiperspirant composition may include a gelling agent typical of antiperspirant compositions. For example, in certain embodiments, the antiperspirant composition may include gelling agents such as, but not limited to, waxes, esters of fatty acid and fatty alcohol, triglycerides, partially or fully hydrogenated soybean oil, partially or fully hydrogenated castor oil, other partial or fully hydrogenated plant oils, stearyl alcohol, or other cosmetically acceptable materials, which are solid or semi solid at room temperature and provide a consistency suitable for application to the skin. The antiperspirant composition may also include a combination of gelling agents. For example, the gelling agent may be a mixture of a high melting point wax(es) and a low melting point wax(es), such as hydrogenated castor oil and stearyl alcohol.

The antiperspirant composition may include between 20 weight % and 30 weight % of a gelling agent, based on a total weight of the antiperspirant composition. For example, the antiperspirant composition may include between 22 weight % and 28 weight % of a gelling agent. In one embodiment, the antiperspirant composition includes about 25 weight % of a gelling agent. For example, the antiperspirant composition may include between 20 weight % and 30 weight of a combination including hydrogenated castor oil, hydrogenated soybean oil, and stearyl alcohol as the gelling agent.

The antiperspirant composition may also include a surfactant. For example, the antiperspirant composition may include one or more surfactants such as alkanolamides (such as N-alkyl pyrrolidone), ethoxylated amides, amine oxides (for example, cocamidopropylamine oxide), ethoxylated fatty acids (for example, PEG-8 distearate), ethoxylated glycerides (for example, PEG-4 castor oil), glycol esters (for example, propylene glycol ricinoleate), monoglycerides (for example, glycerol myristate), polyglyceryl esters (for example, polyglyceryl-4 oleyl ether), polyhydric alcohol esters and ethers (for example, sucrose distearate), sorbitan/sorbitan esters (for example, sorbitan sesquiisostearate), triesters of phosphoric acid (for example, trioleth-8 phosphate), ethoxylated lanolin (for example, PEG-20 lanolin), propoxylated polyoxyethylene ethers (for example, PPG-5 ceteth-20), and alkylpolyglycosides (for example, lauryl glucose).

The surfactant (or surfactant blend) may include non-ionic compounds, and may also include blends thereof with cationic (for example, PEG-15 tallow amine) or anionic (for example, sodium lauroyl laurate) surfactants.

The antiperspirant composition may include from 1% to 15% surfactant, based on a total weight of the antiperspirant composition. Alternatively, the antiperspirant composition may include from 1 weight % to 10 weight % surfactant. In one embodiment, the antiperspirant composition includes from 2 weight % to about 6 weight % surfactant.

The antiperspirant composition may include one or more antiperspirant actives. In some embodiments, the antiperspirant actives are compatible with the other ingredients in the antiperspirant composition.

In one embodiment, the antiperspirant actives includes at least one of aluminum chlorohydrate, aluminum chloride, and aluminum zirconium. For example, the antiperspirant active may be aluminum zirconium tetrachlorohydrex glycine.

In some embodiments, the antiperspirant actives are the only active odor control or sweat prevention ingredient. For example, in one embodiment, the aluminum zirconium tetrachlorohydrex glycine is the only active odor control or sweat prevention ingredient in the antiperspirant composition.

The antiperspirant composition includes an effective amount of antiperspirant actives. For example, the antiperspirant composition may include an amount of antiperspirant actives that is effective to reduce the flow of perspiration in the axillary region. In other embodiments, the antiperspirant composition may include an amount of antiperspirant actives that is effective to reduce malodor or to act as an antibacterial, or prevent sweat.

The antiperspirant composition may include from about 10% to about 30% antiperspirant actives, based on a total weight of the antiperspirant composition. For example, the antiperspirant composition may include from about 10 weight % to about 25 weight % antiperspirant actives or from about 15 weight % to about 23 weight % antiperspirant actives. In one embodiment, the antiperspirant composition includes about 21 weight % antiperspirant actives. For example, the antiperspirant composition may include from about 20.5 weight % to about 21.5 weight % of aluminum zirconium tetrachlorohydrex glycine.

In certain embodiments, the antiperspirant composition includes additional ingredient typical of antiperspirant compositions. For example, the antiperspirant composition may include fragrances, preservatives, antioxidants, colorants, and emulsifiers.

The antiperspirant composition may include one or more preservatives. In some embodiments, the preservatives improve an antimicrobial characteristic of the antiperspirant composition to improve storage life or prevent decay. In other embodiments, the preservatives may also enhance the functional characteristics of the antiperspirant composition. For example, in some embodiments, the preservative may provide deodorant or emollient effects to the antiperspirant composition.

For example, in one embodiment, the one or more preservatives include at least one of phenoxyethanol, caprylyl glycol, ethylhexylglycerin, citric acid, benzoic acid, lactic acid, and combinations thereof. In other embodiments, the one or more preservatives consist essentially of only one of phenoxyethanol, caprylyl glycol, ethylhexylglycerin, citric acid, benzoic acid, or lactic acid, with only trace amounts of other preservative materials. In one example of such an embodiment, the composition contains one preservative, where the preservative consists of at least 990% of only one of phenoxyethanol, caprylyl glycol, ethylhexylglycerin, citric acid, benzoic acid, or lactic acid.

In certain embodiments, the preservative may include one or more antioxidant. For example, antioxidants may be added to the antiperspirant composition to act as ingredient protectants and for maintenance of long-term stability of the composition. Suitable antioxidants include Tinogard, manufactured by Ciba Specialty Chemicals, Basel, Switzerland.

The antiperspirant composition may include an effective amount of preservatives. For example, the antiperspirant composition may include an amount of preservatives effective to reduce a spoilage of the antiperspirant composition during storage or use.

In some embodiments, the antiperspirant composition may optionally include one or more fragrances. A variety of fragrances can be used in the antiperspirant compositions if a scented product is desired. For example, in some embodiments, any fragrance suitable for personal care use may be incorporated into the antiperspirant composition as a non-essential ingredient.

### Examples

Aspects of the present disclosure may be further understood by referring to the following examples. The examples are illustrative, and are not intended to be limiting embodiments thereof. Table 1 illustrates antiperspirant compositions according to embodiments of the present disclosure with comparative measures of white residue against a control formulation.

The compositions of Table 1 were prepared as follows: The emollients were placed in a beaker and heated with stirring. The gelling agents and surfactants were then added. Cyclomethicone or the cyclomethicone replacements (Crodamol STS, Crodamol SFX, Arlamol LST, Tegosoft AC, Tegosoft DEC), were then added. The mixture was then stirred and the antiperspirant active was added. Then the remaining ingredients were added and mixed for one minute. The mixture was then poured into stick containers of the type used for antiperspirants, and placed in a refrigerator for cooling. Cooling was then completed at room temperature.

The compositions of Table 1 were then applied to a surface, and then a colorimetric measurement of the white residue left after application was taken within a few seconds of application. The color change can be measured according to the L*a*b* color scale. The luminance or lightness (L*) value measures brightness and varies from a value of one hundred for perfect white to zero for black, assuming a* and b* are zero. The a* value is a measure of redness when positive, gray when zero and greenness when negative. The b* value is a measure of yellowness when positive, gray when zero and blueness when negative. Generally, white residue is more visible as the L* value increases, meaning the residue is brighter and more noticeable.

The compositions of Table 1 were tested as follows: for each composition of Table 1, a 10.2 cm×12.7 cm strip of black vinyl (manufactured by Uniroyal) was weighed, and a base colorimetric reading (black-white, or L-axis) was obtained using a Gardner XL-800 Tristimulus colorimeter at three places on the strip. The antiperspirant composition was then evenly applied by wiping the gel stick across the vinyl strip until 0.35 grams of the product was deposited. A colorimeter reading was then immediately obtained at three points on the vinyl strip. The average color difference reading (ΔL) was then obtained for each strip by subtracting the average colorimeter reading of the untreated strip from the average reading for the treated strip. (Note that the more positive the ΔL value, the greater the visible white residue left by the product.)

TABLE 1

| | Control | | | | | |
|---|---|---|---|---|---|---|
| Ingredient | Comp. | Comp. 1 | Comp. 2 | Comp. 3 | Comp. 4 | Comp. 5 |
| C12-15 alkyl benzoate, | 15.69% | 15.69% | 15.69% | 15.69% | 15.69% | 15.69% |
| PPG-14 butyl ether, and | | | | | | |
| PEG-8 distearate | | | | | | |
| Hydrogenated castor oil, | 49.91% | 49.91% | 49.91% | 49.91% | 49.91% | 49.91% |
| Hydrogenated soy bean | | | | | | |
| oil, Behenyl alcohol, | | | | | | |
| Stearyl alcohol, and | | | | | | |
| Reodorized PKO | | | | | | |
| 50% citric acid and | 0.06% | 0.06% | 0.06% | 0.06% | 0.06% | 0.06% |
| Tinoguard TTDD | | | | | | |
| Aluminum zirconium | 20.94% | 20.94% | 20.94% | 20.94% | 20.94% | 20.94% |
| tetrahydrex glycine | | | | | | |
| Cyclomethicone | 13.40% | — | — | — | — | — |
| Crodamol STS | — | 13.40% | — | — | — | — |
| Crodamol SFX | — | — | 13.40% | — | — | — |
| Arlamol LST | — | — | — | 13.40% | — | — |
| Tegosoft AC | — | — | — | — | 13.40% | |
| Tegosoft DEC | — | — | — | — | — | 13.40% |
| Total | 100% | 100% | 100% | 100% | 100% | 100% |
| L*(D65) | 46.87 | 35.00 | 38.79 | 42.54 | 41.66 | 39.04 |

CIELAB (L*a*b) L defines lightness

As illustrated in Table 1, exemplary compositions 1-5 had a lower L* value when compared to a control composition which includes cyclomethicone. A lower L* value indicates that there was less white particles or white material on a test strip. Accordingly, replacing cyclomethicone with an emollient as described herein, reduced the amount of white residue left after application.

The present disclosure has been described with reference to exemplary embodiments. Although a limited number of embodiments have been shown and described, it will be appreciated by those skilled in the art that changes may be made in these embodiments without departing from the principles and spirit of preceding detailed description. It is intended that the present disclosure be construed as including all such modifications and alterations insofar as they come within the scope of the appended claims or the equivalents thereof.

What is claimed is:

1. An antiperspirant composition, comprising:
an antiperspirant active;
a surfactant;
a gelling agent; and
an emollient,
wherein the emollient comprises at least one of PPG-3 isostearyl methyl ether, PPG-3 benzyl ether myristate, PPG-3 benzyl ether ethylhexanoate, isoamyl cocoate, and diethylhexyl carbonate, and
wherein the antiperspirant composition comprises 1% or less of cyclomethicone, based on a total weight of the antiperspirant composition.

2. The antiperspirant composition of claim 1, wherein the antiperspirant composition is substantially free of cyclomethicone.

3. The antiperspirant composition of claim 1, comprising:
from 10 weight % to 30 weight % of the antiperspirant active;
from 1.0 weight % to 15 weight % of the surfactant;
from 20 weight % to 30 weight % of the gelling agent; and
from 0.1 weight % to 50 weight % of the emollient.

4. The antiperspirant composition of claim 1, wherein the antiperspirant active comprises aluminum zirconium tetrahydrex glycine.

5. The antiperspirant composition of claim 1, wherein the gelling agent comprises at least one of hydrogenated castor oil, hydrogenated soy bean oil, and stearyl alcohol.

6. The antiperspirant composition of claim 1, wherein the emollient further comprises at least one of C12-15 alkyl benzoate, PPG-14 butyl ether, and palm kernel oil.

7. The antiperspirant composition of claim 1, wherein the surfactant comprises PEG-8 distearate.

8. A cyclomethicone-free antiperspirant composition, comprising:
an antiperspirant active;
a surfactant;
a gelling agent; and
a cyclomethicone replacement emollient,
wherein the cyclomethicone replacement emollient consists essentially of at least one of PPG-3 isostearyl methyl ether, PPG-3 benzyl ether myristate, PPG-3 benzyl ether ethylhexanoate, isoamyl cocoate, diethylhexyl carbonate, and mixtures thereof, and
wherein the antiperspirant composition comprises 1% or less of cyclomethicone, based on a total weight of the antiperspirant composition.

9. The cyclomethicone-free antiperspirant composition of claim 8, wherein the cyclomethicone-free antiperspirant composition comprises 0% cyclomethicone.

10. The antiperspirant composition of claim 9, comprising:
from 10 weight % to 30 weight % of the antiperspirant active;
from 1.0 weight % to 15 weight % of the surfactant;
from 20 weight % to 30 weight % of the gelling agent; and
from 0.5 weight % to 50 weight % of the emollient, and
from 10 weight % to 20 weight % of the cyclomethicone replacement emollient.

11. The antiperspirant composition of claim 8, wherein the cyclomethicone replacement emollient consists essentially of PPG-3 isostearyl methyl ether, consists essentially of PPG-3 benzyl ether myristate, consists essentially of PPG-3 benzyl ether ethylhexanoate, consists essentially of isoamyl cocoate, or consists essentially of diethylhexyl carbonate.

12. A method for decreasing the visibility of an antiperspirant composition after application, the method comprising:
adding a cyclomethicone replacement emollient to the antiperspirant composition,
wherein the cyclomethicone replacement emollient comprises at least one of PPG-3 isostearyl methyl ether, PPG-3 benzyl ether myristate, PPG-3 benzyl ether ethylhexanoate, isoamyl cocoate, and diethylhexyl carbonate, and
wherein the antiperspirant composition comprise 1% or less cyclomethicone, based on a total weight of the antiperspirant composition.

13. The method of claim 12, wherein the antiperspirant is substantially free of cyclomethicone.

14. The method of claim 12, wherein the antiperspirant composition comprises:
from 10 weight % to 20 weight % of the cyclomethicone replacement emollient.

15. The method of claim 12, further comprising:
adding from 10 weight % to 30 weight % of an antiperspirant active to the antiperspirant composition;
adding from 1.0 weight % to 15 weight % of a surfactant to the antiperspirant composition; and
adding from 20 weight % to 30 weight % of the gelling agent to the antiperspirant composition;
wherein adding the cyclomethicone replacement emollient comprises adding from 10 weight % to 20 weight % of the cyclomethicone replacement emollient.
